(19) United States
(12) Patent Application Publication
(10) Pub. No.: US 2016/0206475 A1
(43) Pub. Date: Jul. 21, 2016

(54) OVER-MOLDED GOGGLE FRAME

(71) Applicant: Spy Optic Inc., Carlsbad, CA (US)

(72) Inventors: Brittany Kay Reynolds, Carlsbad, CA (US); Keith Larronde Asher, Encinitas, CA (US)

(21) Appl. No.: 14/992,885

(22) Filed: Jan. 11, 2016

Related U.S. Application Data
(60) Provisional application No. 62/105,627, filed on Jan. 20, 2015.

(51) Int. Cl. A61F 9/02; B29C 45/00

(52) U.S. Cl. CPC A61F 9/025; B29C 45/0003; B29K 2055/02

(57) ABSTRACT

A goggle frame having a stiff first frame member and a flexible second frame member over-molded onto the stiff first frame member. The stiffness of the first frame member allows a goggle lens to be mounted to the first frame member and for the first frame member to control deflection or distortion of the goggle lens. The flexibility of the second frame member allows the second frame member to flex and conform to the unique contour of the wearer's face.

### CROSS-REFERENCE TO RELATED APPLICATIONS

This application claims the benefit of U.S. Provisional Application No. 62/105,627, filed Jan. 20, 2015, the contents of which are expressly incorporated herein by reference.

### STATEMENT RE: FEDERALLY SPONSORED RESEARCH/DEVELOPMENT

Not Applicable

### BACKGROUND

Various aspects of the present disclosure generally relate to a goggle frame, and more specifically to a goggle frame having a stiff frame member and a flexible frame member over-molded onto the stiff frame member.

Goggles are well-known in the art and have been used for a wide variety of reasons. For instance, it is very common for those participating in winter sports, such as snowboarding, skiing, snowmobiling, etc., as well as motor sports, such as motocross, to use a goggle for protective reasons, as well as to enhance the wearer's ability to see. The goggle may protect the wearer's eyes by, forming a barrier around the wearer's eyes to keep debris, such as snow, ice, or dirt from contacting the wearer's eyes. The goggle may additionally make it easier for the user to see by providing a tinted lens, similar to sunglasses. For instance, sunlight may reflect off of snow or ice to create a problematic glare within the wearer's line of sight, and thus, a tinted lens may block some of the glare.

A conventional goggle generally includes a frame and a lens coupled to the frame. A strap is additionally coupled to the frame and is configured for use in securing the goggle to the wearer's head. The strap is typically adjustable to conform to the size of the wearer's head and is disposable in tension to keep the goggle frame and lens in proper position and alignment with the wearer's eyes while the wearer performs the activity (e.g., snowboarding, etc.).

Although the use of a goggle has several advantages, as discussed above, conventional goggles suffer due to flexing of the goggle lens. Along these lines, the tension in the strap may cause the entire goggle frame to flex, which typically causes flexing or distorting of the goggle lens. When the goggle lens is flexed or distorted, the viewing through the lens may become blurry or distorted. The distorted vision may be particularly detrimental when the wearer is required to make split-second decisions based on what the wearer observes, as is the case in the fast-moving sports of snowboarding, skiing, and motocross.

Accordingly, there is a need in the art for a goggle specifically adapted to mitigate tension in the strap being passed to the frame so as to prevent distortion of the goggle lens. Various aspects of the present disclosure are directed toward addressing the foregoing deficiencies in the art.

### BRIEF SUMMARY

The present disclosure generally relates to a goggle frame having a stiff first frame member and a flexible second frame member over-molded onto the stiff first frame member. The stiffness of the first frame member allows a goggle lens to be securely mounted to the first frame member and for the first frame member to control deflection or distortion of the goggle lens. The flexibility of the second frame member allows the second frame member to flex and conform to the unique contour of the wearer's face in response to the tension applied by a goggle strap.

According to one embodiment, there is provided a composite goggle frame for use by a user with a goggle lens and a goggle strap. The composite goggle frame includes a first frame element molded from a first material that is of a first hardness, with the first frame element extending around a first opening adapted to receive the goggle lens. The first frame element is engageable with the goggle lens to retain the goggle lens within the first opening. A second frame element is molded onto the first frame element from a second material that is of a second hardness less than the first hardness. The second frame element extends around a second opening aligned with the first opening so that at least a portion of the second opening overlaps with a portion of the first opening. The second frame element includes opposed end portions configured to engage with respective portions of the goggle strap to enable securement of the goggle frame to the user.

The first frame element may completely circumnavigate the first opening. The first frame element may comprise an upper portion, a lower portion and a pair of opposed lateral portions spaced from each other and extending between the upper portion and the lower portion, with the upper portion, lower portion and pair of opposed lateral portions collectively circumnavigating the first opening. The first frame element may further comprise at least one lens attachment element adapted to engage with the lens for securing the lens to the first frame element. The first frame element may be formed from a polycarbonate-acrylonitrile butadiene styrene (ABS) blend.

The second frame element may include a pair of loops adapted to engage with respective portions of the goggle strap. The second frame element may be adapted to interface with the user and be positioned between the user and the first frame element during use of the goggle frame. The second frame element may be formed of Thermoplastic Polyurethane 85. The second frame element may completely circumnavigate the second opening.

The second frame element may include a primary wall positioned about the second opening and a plurality of flexible support elements extending between the primary wall and the first frame element.

According to another embodiment, there is provided a composite goggle frame for use with a goggle lens, wherein the composite goggle frame includes a rigid frame element extending at least partially around a first opening, and being specifically configured and adapted to be engageable with the goggle lens when the goggle lens is positioned in the first opening. A flexible frame element is coupled to the rigid frame element and extends at least partially around a second opening at least partially overlapping the first opening. The flexible frame element is adapted to flex in response to a load being applied thereto so as to reduce transfer of forces from the flexible frame element to the rigid frame element.

According to one embodiment, the first/rigid frame member is formed of a first material having a stiffness sufficient to mount the goggle lens thereto and to mitigate deflection of the goggle lens. The second/flexible frame member is over-molded onto the first frame member and is formed of a second material having a stiffness less than the first frame member.

According to yet another embodiment, there is provided a method of forming a composite goggle frame adapted for use with a goggle lens and a goggle strap. The method includes molding a first frame element from a first material that is of a first hardness, with the first frame element being molded so as to extend around a first opening adapted to receive the goggle lens and to be engageable with the goggle lens to retain the goggle lens within the first opening. The method further includes molding a second frame element onto the first frame element from a second material that is of a second hardness less than the first hardness, with the second frame element extending around a second opening aligned with the first opening so that at least a portion of the second opening overlaps with a portion of the first opening, the second frame element having opposed end portions configured to engage with respective portions of the goggle strap to enable securement of the goggle frame to the user.

The step of molding the first frame element and the step of molding the second frame element may occur in a common mold. The step of molding the first frame element may occurs in a first portion of the common mold and the step of molding the second frame element may occur in a second portion of the common mold.

The step of molding the first frame element may occur in a first mold and the step of molding the second frame element may occur in a second mold different from the first mold. The method may further include the step of transferring the first frame element from the first mold to the second mold.

The step of molding the second frame element occurs after at least a portion of the first frame element is formed.

According to another embodiment, there is provided a method of forming a goggle frame. The method includes the steps of providing a mold having a first portion corresponding to a first frame member and a second portion corresponding to a second frame member. A first material is injected into the first portion to form a first frame member and a second material is injected into the second portion to Rum the second frame member, with the second frame member being over-molded onto the first frame member.

The present disclosure will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a front perspective view of a goggle frame constructed in accordance with an embodiment of the present disclosure;

FIG. 2 is a rear perspective view of the goggle frame depicted in FIG. 1;

FIG. 3 is a rear exploded perspective view of the goggle frame depicted in FIGS. 1 and 2;

FIG. 4 is a flow chart related to a first embodiment of a method of forming a composite goggle frame; and

FIG. 5 is a flow chart related to a second embodiment of a method of forming a composite goggle frame.

Common reference numerals are used throughout the drawings and the detailed description to indicate the same elements.

### DETAILED DESCRIPTION

The detailed description set forth below in connection with the appended drawings is intended as a description of certain embodiments of an over-molded goggle frame and is not intended to represent the only forms that may be developed or utilized. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual relationship or order between such entities.

Referring now to the drawings wherein the showings are for purposes of illustrating a preferred embodiment of the present disclosure only, and are not for purposes of limiting the same, there is depicted a goggle frame 10 generally including, a first frame member 12 that is of a first stiffness and a second frame member 14 over-molded onto the first frame member 12, wherein the second frame member 14 is of a second stiffness less than the first stiffness. As will be described in more detail below, the stiffer first frame member 12 may be specifically configured and adapted for mounting a goggle lens 15 therein, while the more flexible second frame member 14 may be specifically configured and adapted to conform to the particular contour of the wearer's face when the goggle is secured thereto via a strap (not shown). In this regard, the more flexible second frame member 14 effectively acts as a shock absorber to isolate the stiffer first frame member 12 from stresses imparted by tension in the strap. By isolating the more rigid frame member 14 from the strap tension, the lens 15 may remain in its natural, arcuate configuration, thereby mitigating optical distortion in the lens 15.

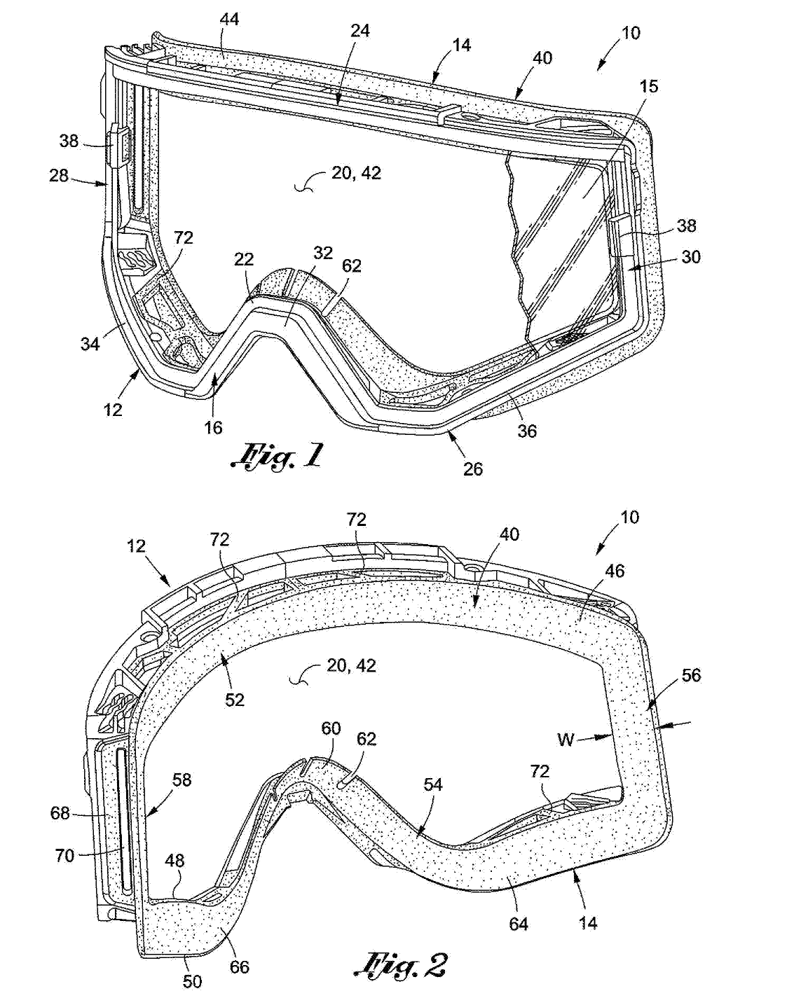

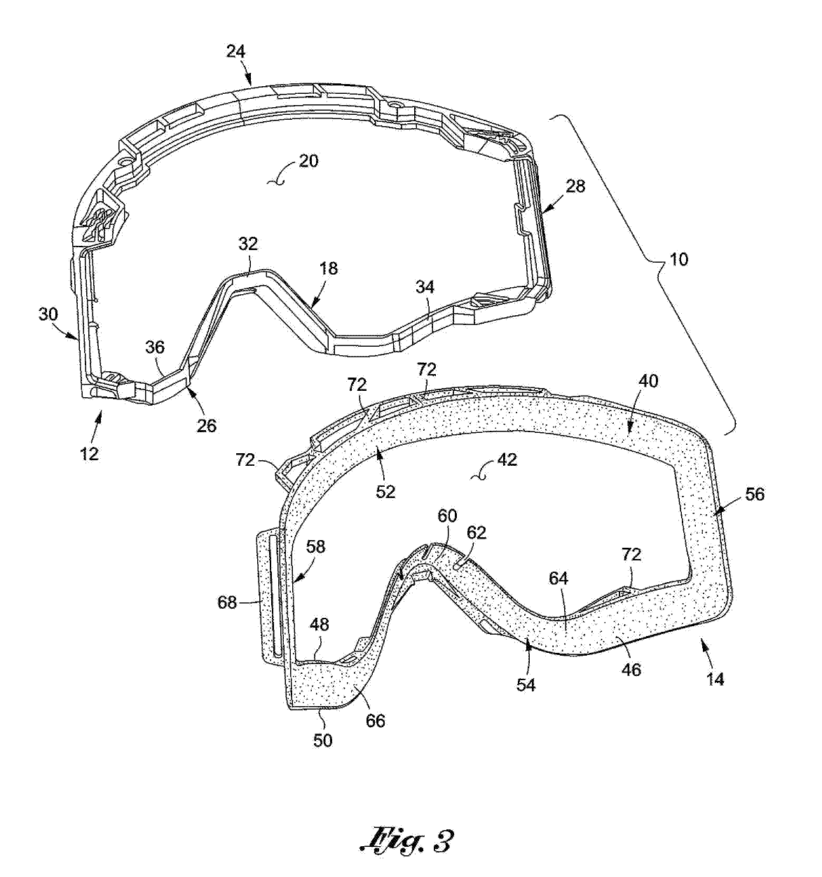

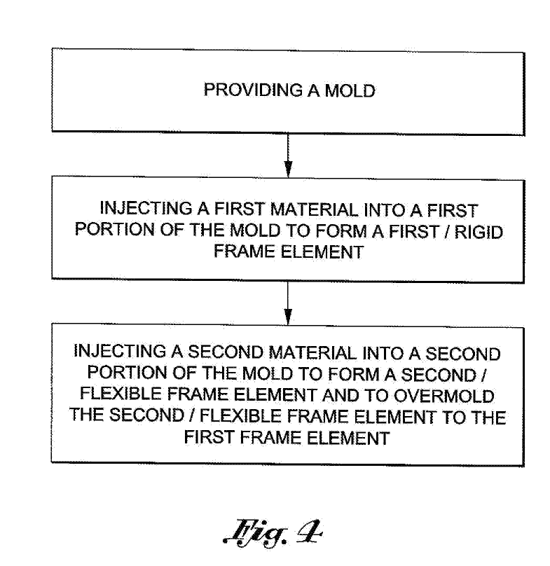

An exemplary goggle frame 10 is depicted in FIGS. 1-3, wherein FIG. 1 is a front perspective view of the goggle frame 10, FIG. 2 is a rear perspective view of the goggle frame 10 and FIG. 3 is an exploded rear perspective view of the goggle frame 10. FIG. 1 includes a partial cutaway view of the lens 15 for purposes of clarity. It is contemplated that the goggle frame 10 may be adapted for use with a conventional goggle lens 15 and a goggle strap for attaching the goggle to the user. The goggle strap is not depicted in FIGS. 1-3 in order to more clearly illustrate the goggle frame 10.

The first frame member 12 (e.g., the stiffer frame member) includes a front surface 16 (see FIG. 1) and an opposing rear surface 18 (see FIG. 3). The front surface 16 is generally convex, while the rear surface 18 is generally concave. Both the front and rear surfaces 16, 18 extend around a central opening 20. A lens engagement surface 22 is located between the front surface 16 and the rear surface 18 and is adapted to be engageable with the goggle lens 15, which when coupled to the first frame member 12, extends from the lens engagement surface 22 toward the front surface 16, with the outer surface of the lens 15 preferably being flush with the front surface 16 of the first frame member 12.

The first frame member 12 additionally includes an upper portion 24, a lower portion 26 and a pair of opposed lateral portions 28, 30. The lower portion 26 include a bridge section 32 adapted to extend over a user's nose, wherein the bridge section 32 is interposed between a pair of connecting sections 34, 36, which extend between the bridge section 32 and respective ones of the opposed lateral portions 28, 30.

The first frame member 12 may further include one or more lens attachment members 38. As shown in FIG. 1, the lens attachment members 38 include a pair of clips or tabs which may be advanced through corresponding openings formed in the lens 15. It is contemplated that the goggle frame 10 and the corresponding lens 15 may be adapted to achieve a substantially permanent connection between the goggle frame 10 and the lens 15, such that the lens 15 remains attached to the goggle frame 10 throughout the lifespan of the goggle. Alternatively, it is contemplated that the goggle frame 10 and lens 15 may be adapted to enable selective detachable engagement between the goggle frame 10 and the lens 15. Along these lines, it understood that when the goggle is worn, the environmental conditions may change. For instance, when the goggle is worn when skiing or snowboarding, clear skies in one part of the day may result in the sun producing glare off the snow, which may lead to bright conditions. As such, the user may want a darker lens to block some of the glare or brightness. However, clouds may roll in later in the day producing overcast, dark conditions, which are not suitable to the darker lens. Consequently, the user may want to swap the darker lens for a lighter lens. In that case, the goggle frame 10 may include a retaining element which may be transitioned between locked and unlocked positions for purposes of either retaining the lens 15 (e.g., the locked position) or removing the lens 15 (e.g., the unlocked position). An exemplary frame 10 which can accommodate such lens-changing functionality is shown and described in U.S. Provisional Patent Application Ser. No. 62/064,842 filed Oct. 16, 2014 and entitled Goggle Lens Changing System, and its counterpart U.S. Non-Provisional Patent Application Ser. No. 14/882,789, filed Oct. 14, 2015, both of which are co-owned by the Applicant of the present application, and the contents of which are expressly incorporated herein by reference.

The first frame member 12 is fabricated from a material that is of a first stiffness, which is stiffer and more rigid than the second frame member 12, as will be discussed in more detail below. The first frame member 12 may be fabricated from a polymeric material, such as a polycarbonate—acrylonitrile butadiene styrene (ABS) blend, or other materials known by those skilled in the art.

The second frame member 14 is molded onto the first frame member 12, wherein the second frame member 14 includes a primary wall 40 which circumnavigates an opening 42, which overlaps the opening 20 formed by the first frame member 12, with the opening 42 being specifically sized and adapted to fit around both of the wearer's eyes. The primary wall 40 includes an outer surface 44 (see FIG. 1) and an opposing inner surface 46 (see FIGS. 2 and 3). The outer surface 44 defines a generally convex configuration, while the inner surface 46 defines a generally concave configuration. The inner surface 46 may be covered by a foam or other compressible material for creating a seal or barrier around the wearer's eyes. The primary wall 40 defines an inner periphery 48 and an outer periphery 50, the second frame member 14 having a width, “W” equal to the distance between the inner periphery 48 and the outer periphery 50.

The primary wall 40 includes an upper portion 52, a lower portion 54, and a pair of opposed lateral portions 56, 58, which generally correspond to the upper portion 24, lower portion 26, and pair of opposed lateral portions 28, 30 of the first frame member 12, respectively. The lower portion 54 includes a bridge section 60 adapted to extend over a user's nose. Along these lines, the bridge section 60 may include one or more slits 62 which enhance the ability of the bridge section 60 to conform to the particular contour of the wearer's nose. The bridge section 60 is interposed between a pair of connecting sections 64, 66, which extend between the bridge section 60 and respective ones of the opposed lateral portions 56, 58.

The second frame member 14 further includes a pair of lateral elements 68, which extend laterally outward from respective ones of the lateral portions 56, 58. Each lateral element 68 includes a slit 70 adapted to accommodate a goggle strap. In this respect, the size of the slit 70 may correspond to the size and shape of the goggle strap.

The second frame member 14 additionally includes a plurality of flexible support elements 72 coupled to the primary wall 40 and extending toward the first frame member 12. The flexible support elements 72 are adapted to allow the primary wall 40 to move or flex relative to the first frame member 12 when the goggle frame 10 is worn by the wearer. The goggle frame 10 may include upper flexible support members, lower flexible support members, as well as support members located on lateral sides of the goggle frame 10. Each flexible support element 72 may be transitional between a flexed configuration and an unflexed configuration, wherein at least a portion of the primary wall 40 moves toward the first frame member 12 as the flexible support element 72 transitions from the unflexed configuration toward the flexed configuration.

According to one embodiment, the second frame member 14 is fabricated from Thermoplastic Polyurethane 85 (e.g., TPU 85), although other materials known in the art may also be used without departing from the spirit and scope of the present disclosure.

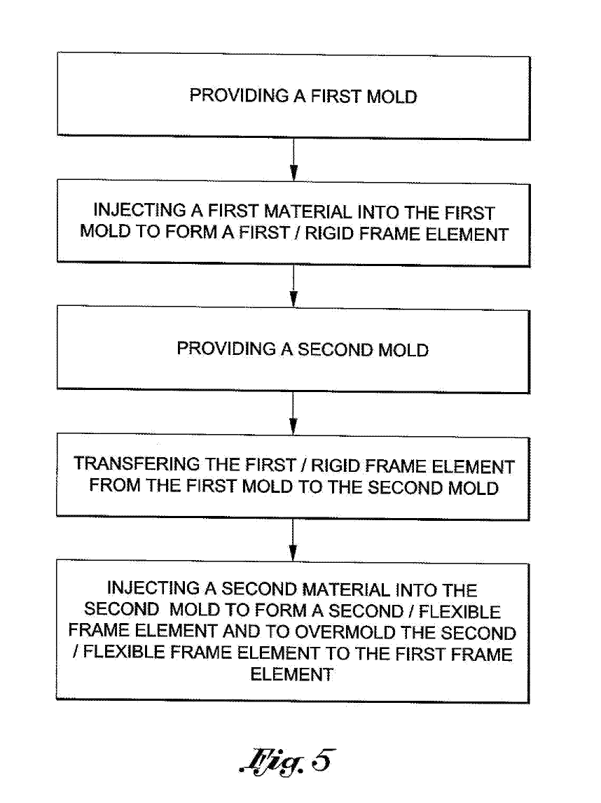

One of the unique aspects of the present disclosure is the method by which the goggle frame 10 is fabricated, particularly, that the second frame member 14 is over-molded onto the first frame member 12. According to one embodiment, and referring to the flow chart of FIG. 4, a single mold is used to create both the first frame member 12 and the second frame member 14. wherein a first portion of the mold is configured to form the first frame member 12, and a second portion of the mold is configured to form the second frame member 14. A first injection port is used to inject the first material into the first portion of the mold for forming the first frame member 12. A second injection port is used to inject the second material into the second portion of the mold for forming the second frame member 14. As noted above, the first material has a stiffness that is greater than the stiffness of the second material. The first material stiffness provides the necessary rigidity or firmness to control deflection of the goggle lens 15, while the second stiffness enables the second frame member 14 to conform to the unique contours of the wearer's face.

According to one implementation of the disclosure, the first frame member 12 is at least partially formed before the second material is injected into the mold. In this respect, the first material is injected into the mold and is allowed to partially harden before the second material is injected into the mold. Since the second frame member 14 is over-molded onto the first frame member 12, the second frame member 14 is attached to the first frame member 12 without any intervening coupling elements. Rather, once the goggle frame 10 is formed, the first and second frame members 12, 14 operate as a single, integral unit.

Although the foregoing describes an embodiment wherein the first and second frame members 12, 14 are formed in a single mold, it is understood that in other embodiments, such as the method related to the flow chart of FIG. 5, the first and second frame members 12, 14 are formed in separate molds. For instance, the first material may be injected into a first mold and allowed to harden to form the first frame member 12. The first frame member 12 may then be removed from the first mold and transferred to the second mold, wherein the first frame member 12 is subsequently used as an insert in a second mold into which the second material may be injected for over-molding the second frame member 14 onto the first frame member 12. After the second material hardens to form the second frame member 14, the goggle frame 10 is removed from the second mold.

The softer second material allows the second frame member 14 to be used as a buffer of shock absorber which absorbs the tightening forces used for cinching/tightening the goggle strap onto the wearer's head. Along these lines, when the goggle is placed on the wearer's head, and the goggle strap is tightened, the tension in the goggle strap will cause the second frame member 14 to flex or deflect to conform to the shape of the wearer's head. The deflection of the second frame member 14 mitigates the transfer of the forces to the first frame member 12. In this respect, the second frame member 14 at least partially isolates the first frame member 12 from the tension forces in the strap. Furthermore, the rigidity of the first frame member 12 allows the first frame member 12 to retain its natural shape, which in turn mitigates deflection of the goggle lens 15.

The particulars shown herein are by way of example only for purposes of illustrative discussion, and are not presented in the cause of providing what is believed to be most useful and readily understood description of the principles and conceptual aspects of the various embodiments of the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A composite goggle frame for use by a user with a goggle lens and a goggle strap, the composite goggle frame comprising:
a first frame element molded from a first material that is of a first hardness, the first frame element extending around a first opening adapted to receive the goggle lens, the first frame element being engageable with the goggle lens to retain, the goggle lens within the first opening; and
a second frame element molded onto the first frame element from a second material that is of a second hardness less than the first hardness, the second frame element extending around a second opening aligned with the first opening so that at least a portion of the second opening overlaps with a portion of the first opening, the second frame element having opposed end portions configured to engage with respective portions of the goggle strap to enable securement of the goggle frame to the user.

2. The composite goggle frame recited in claim 1, wherein the first frame element completely circumnavigates the first opening.

3. The composite goggle frame recited in claim 1, wherein the first frame element comprises an upper portion, a lower portion and a pair of opposed lateral portions spaced from each other and extending between the upper portion and the lower portion, the upper portion, lower portion and pair of opposed lateral portions collectively circumnavigating the first opening.

4. The composite goggle frame recited in claim 3, wherein the first frame element further comprises at least one lens attachment element adapted to engage with the lens for securing the lens to the first frame element.

5. The composite goggle frame recited in claim 1, wherein the first frame element formed from a polycarbonate-acrylonitrile butadiene styrene (ABS) blend.

6. The composite goggle frame recited in claim 1, wherein the second frame element includes a pair of loops adapted to engage with respective portions of the goggle strap.

7. The composite goggle frame recited in claim 1, wherein the second frame element is adapted to interface with the user and be positioned between the user and the first frame element during use of the goggle frame.

8. The composite goggle frame recited in claim 1, wherein the second frame element is formed of Thermoplastic Polyurethane 85.

9. The composite goggle frame recited in claim 1, wherein the second frame element includes a primary wall positioned about the second opening and a plurality of flexible support elements extending between the primary wall and the first frame element.

10. The composite goggle frame recited in claim 1, wherein the second frame element completely circumnavigates the second opening.

11. A composite goggle frame for use with a goggle lens, the composite goggle frame comprising:
a rigid frame element extending at least partially around a first opening and being specifically configured and adapted to be engageable with the goggle lens when the goggle lens is positioned in the first opening; and
a flexible frame element coupled to the rigid frame element and extending at least partially around a second opening at least partially overlapping the first opening. the flexible frame element being adapted to flex in response to a load being applied thereto so as to reduce transfer of forces from the flexible frame element to the rigid frame element.

12. The composite goggle frame recited in claim 11, wherein the rigid frame element is formed of a first material that is of a first hardness and the flexible frame element is formed of a second material that is of a second hardness less than the first hardness.

13. The composite goggle frame recited in claim 11, wherein the first frame element formed from a polycarbonate-acrylonitrile butadiene styrene (ABS) blend.

14. The composite goggle frame recited in claim 11, wherein the second frame element is formed of Thermoplastic Polyurethane 85.

15. A method of forming a composite goggle frame adapted for use with a goggle lens and a goggle strap, the method comprising the steps of:
molding a first frame element from a first material that is of a first hardness, the first frame element being molded so as to extend around a first opening adapted to receive the goggle lens and to be engageable with the goggle lens to retain the goggle lens within the first opening; and
molding a second frame element onto the first frame element from a second material that is of a second hardness less than the first hardness, the second frame element extending around a second opening aligned with the first opening so that at least a portion of the second opening, overlaps with a portion of the first opening, the second frame element having opposed end portions configured to engage with respective portions of the goggle strap to enable securement of the goggle frame to the user.

16. The method recited in claim 15, wherein the step of molding the first frame element and the step of molding the second frame element occur in a common mold.

17. The method recited in claim 16, wherein the step of molding the first frame element occurs in a first portion of the common mold and the step of molding the second frame element occurs in a second portion of the common mold.

18. The method recited in claim 15, wherein the step of molding the first frame element occurs in a first mold and the step of molding the second frame element occurs in a second mold different from the first mold.

19. The method recited in claim 18, further comprising the step of transferring the first frame element from the first mold to the second mold.

20. The method recited in claim 15, wherein the step of molding the second frame element occurs after at least a portion of the first frame element is formed.
